(19) United States
(12) Patent Application Publication
(10) Pub. No.: US 2006/0264804 A1
(43) Pub. Date: Nov. 23, 2006

(54) Device and kit for delivery of encapsulated substances and methods of use thereof

(76) Inventors: Yoram Karmon, Petah Tikva (IL); Nurit Harel, Tel Aviv (IL); Daniela Mavor, Tel Aviv (IL); Michal Shahar, Rishon Lezion (IL)

Correspondence Address: KENYON & KENYON LLP, 1500 K STREET N.W., SUITE 700, WASHINGTON, DC, 20005, US

(21) Appl. No.: 11/397,864

(22) Filed: Apr. 5, 2006

Related U.S. Application Data
(60) Provisional application No. 60/669,001, filed on Apr. 7, 2005.

(51) Int. Cl. A61N 1/30

(52) U.S. Cl. 604/20

(57) ABSTRACT

The present invention is of a kit and electricity generating device for delivery of an encapsulated composition or mixture of compositions, including an electricity generating device and at least one composition in an encapsulated carrier vehicle which is contacted or applied to the electricity generating device. In some embodiments the device includes a means for maintaining stability of the encapsulated composition prior to bio-membrane penetration. The present invention also provides methods of use thereof. Furthermore, the present invention provides a device and method for delivering a fixed ratio of compounds from a mixture to a bio-membrane comprising the step of electro-transporting an encapsulated mixture of compounds. Still further, the present invention is of a device and method for delivering each compound of a mixture of compounds to the same penetration depth.

### CROSS-REFERENCE TO RELATED APPLICATIONS

This application claims priority to U.S. Provisional Application Ser. No. 60/669,001, filed Apr. 7, 2005 and incorporated by reference herein in its entirety.

### FIELD AND BACKGROUND OF THE INVENTION

The present invention relates to transdermal and/or intradermal drug delivery devices, kits and methods of use for delivery of encapsulated substances. More particularly, the present invention is directed to electrical current driven delivery of micro and nano-encapsulated substances.

The background art includes disclosure of a plurality of encapsulated systems, such as, but not limited to vesicles, rigid vesicles, elastic vesicles, liposomes, niosomes, proniosomes, transfersomes®, ethosomes, L-595-PEG-8-L vesicles, nanoemulsions, nanosomes and nanoparticles as potential vehicles for the delivery of cosmetics and the optimized disposition of active ingredients in particular skin layers.

Liposomes (lipid vesicles) are spherical lipid bilayers that have the ability to encapsulate a solvent, which is freely disposed in the interior. Liposomes enable water soluble and water insoluble materials to be used together in a formulation without the use of surfactants or other emulsifiers. Liposomes do not always fully penetrate the skin, but remain trapped in the stratum corneum. Elastic vesicles such as transfersomes® and ethosomes were developed in order to deliver drugs deeper into the skin.

There is numerous technical and patent literature directed to the delivery of substances, both pharmaceuticals and cosmetics, such as drugs and other beneficial agents, into or through intact skin surfaces by passive processes such as diffusion and osmosis and by active processes such as electrically induced electro-transportation, which includes, but is not limited to iontophoresis, electrophoresis, electroosmosis and/or electroporation. Hereunder, the term “electro-transportation” will collectively represent any of the terms iontophoresis, electrophoresis, electroosmosis and/or electroporation, and the term “electro-transported” will encompass the respective adjectives.

Indeed, there is an extensive list of pharmaceutical substances that are routinely administered transdermally and/or intradermally and a similarly long list of devices and methods known in the art for administering same. Although many substances are administered passively via a dermal patch, there are also many substances that are electrically delivered intradermally or transdermally.

The technique of iontophoresis is in wide use today in the administration of drugs as it effectively delivers electrically charged medicaments through the skin and into the capillary structure and lymphatic system. This technique avoids the gastrointestinal side effects sometimes associated with orally ingested drugs and is preferable to subcutaneous injection because of its relatively benign and painless nature.

Another technique, known as electroporation, facilitates the transdermal or intradermal delivery of uncharged substances by electrically inducing the formation of transient dermal micropores that allow mobilization of the uncharged substances by diffusion.

Accordingly, iontophoresis, as well as other electrically induced techniques, such as electroporation, has been incorporated into many transdermal delivery devices, including a dermal patch. Latent deficiencies of devices for and methods of iontophoretic delivery of active compositions known in the art, include non-optimal penetration of the active ingredient, delivery of a non-fixed ratio of substances from a mixture of substances, instability of water soluble compositions, competition between mixtures of active ingredients and inability to delivery immiscible mixtures.

There is thus a widely recognized need for, and it would be highly advantageous to have, a device, kit or system which facilitates electro-transportation of mixtures of hydrophilic and/or hydrophobic components in fixed ratios to the same penetration depth, stabilization of fragile components, optimal delivery of mixtures of active ingredients and increased penetration of the active ingredient/s such as is provided by the present invention.

### BRIEF DESCRIPTION OF THE DRAWINGS

With specific reference now to the drawings in detail, it is stressed that the particulars shown are by way of example and for the purposes of illustrative discussion of the preferred embodiment of the present invention only, and are presented in the cause of providing what is believed to be the most useful and readily understood description of the principles and conceptual aspects of the invention. In this regard, no attempt is made to show structural details of the invention in more detail that is necessary for a fundamental understanding of the invention, the description taken with the drawings making apparent to those skilled in the art how the several forms of the invention may be embodied in practice.

In the drawings:

FIG. 1 is a sectional view of a fully integrated electricity generating device for delivery of an encapsulated formulation according to an embodiment of the present invention.

FIG. 2 is a schematic view of an exemplary power source according to an embodiment of the present invention.

FIG. 3 is a flow chart of a method of using the device of the present invention according to an embodiment of the present invention.

FIG. 4 is a flow chart of a method of delivery of a fixed ratio of compounds from a mixture of compounds.

FIG. 5 is a graphical representation of the amount of MAP (Magnesium L-Ascorbyl-2-Phosphate) delivered into the skin from encapsulated MAP (whitesphere) compared from non-encapsulated MAP.

### DESCRIPTION OF THE PREFERRED EMBODIMENTS

The present invention is of kits, electricity generating devices and methods of use which can be used for transdermal and/or intradermal delivery of at least one encapsulated substance, such as pharmaceuticals, cosmetics and cosmeceuticals, by any suitable means, such as by electro-transportation, iontophoresis, electrophoresis and/or electroporation. An electricity generating device according to the present invention facilitates increased penetration of active substances, delivery of immiscible substances and delivery of a mixture of oppositely charged substances. Further, the present invention provides a method of achieving delivery of a fixed ratio of compounds from a mixture of compounds to the same penetration depth. An electricity generating device of the present invention is advantageously versatile in the sense that a single device, such as, but not limited to a single patch is operative for increased penetration using transdermal and/or intradermal delivery of a versatile range of substances and/or dosages and the patch may be simply administered by the subject.

Before explaining at least one embodiment of the invention in detail, it is to be understood that the invention is not limited in this application to the details of construction and the arrangement of the components set forth in the following drawings and description. The invention is applicable to other embodiments or of being practiced or carried out in various ways. Also, it is to be understood that the phraseology and terminology employed herein is for the purpose of description and should not be regarded as limiting.

The principles and operation of a device and kit for transdermal and intradermal delivery of at least one encapsulated substance to a bio-membrane region of a subject according to the present invention many be better understood with reference to the figures and accompanying description.

The Device

In some embodiments, the device of the present invention can be an electricity generating device. Non-limiting examples of suitable electricity generating devices include, but are not limited to an electro-transportation device, iontophoresis device, a bio-membrane patch, a dermal patch, a galvanic stimulation device, an electrokinetic device, an electroporation device, an ultrasound device, a microneedle, a TENS device and a combination thereof. In some embodiments, device is a thin and flexible patch.

In one embodiment, the electricity generating device is a biological membrane patch. The term ‘bio-membrane’ or ‘biological membrane’ as used herein includes any biological or physiological tissue or other material, by which the body prevents free penetration and dissipation of external substances. The term includes a barrier membrane. Examples include, but are not limited to skin, mucosa, nail, hair, vaginal membrane and rectal membrane and combinations thereof. The term ‘biological membrane device’ as used herein refers to an electricity generating device, such as a patch for application to a biological membrane.

Certain features of the device of the present invention are the same regardless of whether the device is a fully integrated device or a device that is part of a kit, such as, but not limited to the substrate base layer/frame, cathode, anode, power source, battery and electrode connections, liner and battery cover, active composition, encapsulation means and means for maintaining encapsulation vesicle stability, which are described hereinbelow.

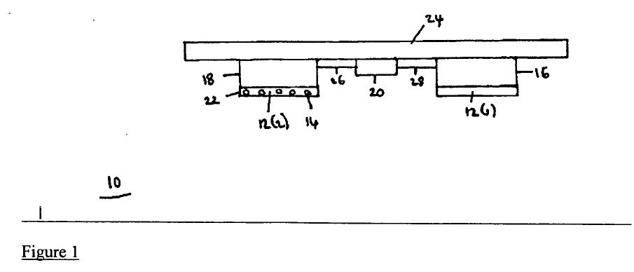

FIG. 1 shows one exemplary device according to one embodiment of the present invention. FIG. 1 shows a schematic view of an electricity generating device configured as a fully integrated patch device according to one embodiment of the present invention. The patch device 10 is fully integrated in the sense that the conductive substance/layer 12 and encapsulated active substance/s 14 are incorporated into the device. In this embodiment, patch 10 may comprise first electrode 16, identified as “cathode,” second electrode 18, identified as “anode”, electrochemical cell 20 as the power source of patch 10, at least one holding means/retainer/separator 22 for accommodating a conductive substance 12, at least one encapsulated active substance 14 and at least one conductive substance 12 (1), 12 (2). Optionally, patch 10 may include a plurality of cathodes 16, a plurality of anodes 18 and a plurality of power supplies 20. In some embodiments, patch 10 may comprise conductive substance/s 12(1), 12(2) to provide an interfacing layer between patch 10 and a body area of a subject.

As shown in FIG. 1, electrodes 16, 18, retainer/holding means 22, conductive substance 12(1), 12(2), encapsulated active substance 14 and electrochemical cell 20 may be supported on a base layer substrate 24. Electrode 16 may be disposed in any suitable way on substrate 24 in spaced relation to electrochemical cell 20 and electrode 18 to define a gap between the two electrodes 16, 18. Optionally, the same conductive layer 12 can be disposed on both anode 18 and cathode 16 or conductive layers 12(1) and 12(2) can be different conductive substance layers. In some embodiments, a hydrogel 12 is disposed in/on the holding means/retainer 22 on the main active electrode and any suitable conductive substance, which can facilitate providing an adhesive conductive interface, is disposed on the counter electrode. In some embodiments, hydrogel 12 is an aqueous hydrogel. In some embodiments, a hydrogel is disposed on the main active electrode and no conductive substance is disposed on the counter electrode.

In some embodiments, patch 10, including patch components, is thin and flexible, to suit the contour of a body area of a subject. In some embodiments, patch 10 is electrically assisted. Patch may optionally be any size, color and shape suitable for application to a desired body area. The thickness of patch 10 can be in some embodiments up to about 10 mm to ensure flexibility, but may be thicker, depending on the application. In one embodiment, thickness of patch 10 can be up to 2 mm. The thickness of the patch may also be dependent upon the type of material used and the flexibility of that material. Patch 10 is in some embodiments disposable, but may be fully or partially reusable. Patch 10 is stable to a wide range of temperatures and humidity. In some embodiments patch can be biocompatible. Patch 10 can be configured to be used on any suitable bio-membrane on any suitable area of the body, including, but not limited to face, neck, arms, hands, legs, thighs, buttocks, feet, toes, fingers, nails, teeth, palms, soles, back, shoulders and torso and combinations thereof.

Detailed description of the patch components, such as the power source, device electrodes, conductive fluid, retainer, active substance, encapsulating vehicles and attachment means are described herein in the relevant subsections.

Patch 10 may further include a skin attachment mechanism, which may be an adhesive layer (not shown in FIG. 1) which serves for attaching patch 10 to a bio-membrane region of the subject, such as, but not limited to an adhesive hydrogel. Adhesive layer can cover at least a portion of bottom surface of patch 10. Adhesive layer may include a biocompatible permeable pressure sensitive adhesive such as Bio-PSA from Dow Corning. Other examples of biocompatible adhesives will be readily apparent to those of ordinary skill in the art. Adhesive layer may be useful for either a single attachment or repeated attachments. Other non-limiting examples of skin attachment mechanisms include clips, elastic, suction means and Velcro and combinations thereof.

In some embodiments, patch 10 of the present invention may be supplied within a protective removable or reusable package, or liner, or cover, so as to provide physical protection and prolong shelf life prior to use. In some embodiments, patch further comprises a release liner (not shown in FIG. 1), which is removed to facilitate attachment, by attachment means. In some embodiments release liner is disposed on the conductive substance layer. In some embodiments, release liner is made from polyester film, wherein one side of release liner can be silicon coated.

In some embodiments, the device of the present invention can be made by a printing technology. In some embodiments, the electrodes, power supply, conductive substance, encapsulated active substance and connections of the device of the present invention can be made and disposed on the device by a suitable printing technique. In some embodiments, the device of the present invention can be a substantially fully printed device.

The Electrode/s

Device, e.g., patch 10 in FIG. 1, may include electrodes referred to hereinafter as ‘anode’ and ‘cathode’, or ‘positive electrode’ and ‘negative electrode’, or ‘active electrode’/‘medical electrode‘/’main electrode’ and ‘counter electrode’, or as ‘device electrodes’, each of which is in electrical contact with power source. Device electrodes can be electrically connected to power source using any well known means, e.g., printed flexible circuits, metal foils, wires, electrically conductive adhesives or by direct contact or a combination thereof. In some embodiments, contact between the electrodes is avoided and the electrodes are in spaced relation to each other. Contact between the two device electrodes can be avoided by the optional use of an insulating element.

In some embodiments, device electrodes are electrically conductive and may be formed of a metal, such as, but not limited to a metal foil or metal deposited or painted on a suitable backing. In some embodiments the device electrode is made from a metal, which has medicinal and/or cosmetic properties, such as, but not limited to silver, zinc, copper, lithium or a combination thereof. Other examples of suitable metals for electrodes include copper, manganese dioxide, aluminum, graphite, nickel, silver/silver chloride, platinum, stainless steel, gold, titanium, or a combination thereof. Alternatively, electrodes may be formed of a hydrophobic polymer matrix containing a conductive filler such as a metal powder/flakes, powdered graphite, carbon fibers, or other known electrically conductive filler material. Any other conductive element or compound, including metal and non-metal materials, can be incorporated into the material of the electrodes. In some embodiments, the electrodes may be provided as thin sheets coupled to the power source, or may be printed onto the base member in spaced relation to each other to define the gap therebetween. In some embodiments at least one electrode is an active electrode and at least one electrode is a counter electrode. Optionally, the active electrode can be the cathode or anode or both the cathode and the anode. Defining which electrode is the active electrode can be dependent on the charge of the composition (e.g., formulation or encapsulation vesicle) being used.

Optionally, the electrode area can be continuous, or formed in any shape or configuration. Optionally, each electrode may not have the same shape and/or same area.

Optionally, electrodes may be in any suitable conformation in relation to each other including but not limited to a coplanar and cofacial arrangement. In some embodiments, electrodes are in a conformation, which readily facilitates diffuse area treatment. In some embodiments, electrodes are configured to provide surface treatment and/or dermal treatment of the body area.

Optionally, the device can include a plurality of electrodes comprised of equal or unequal numbers of anodes and cathodes. Such a multi-electrode patch facilitates providing simultaneously a plurality of treatments in different areas with one encapsulated composition or a plurality of different encapsulated compositions in different body areas or the same body area.

In some embodiments electrodes can be applied to device using a printing technology, such as but not limited to, silk print, offset print, jet printing, lamination, materials evaporation or powder dispersion. It is appreciated that each of device electrodes may be of any size and shape, and located with respect to one another, in any arrangement, as may be required to cover the bio-membrane region being treated.

The Power Source

Any power source, e.g. power supply 20 in FIG. 1, of any suitable size or shape, which provides an electrical potential of between about 0.1 Volt and about 100 Volt can be used according to the present invention. In some embodiments, power source is an electrical battery, providing an electrical potential of between about 0.5 Volt and 12 Volts. In some embodiments power source, can supply a voltage of 1.5 V or 3V.

In some embodiments, power source is thin and flexible. In some embodiments, power source thickness should not exceed 4 mm and in some embodiments, power source thickness should be less than 2 mm. In some embodiments, power source is at least one electrochemical cell. The term ‘electrochemical cell’ as used herein includes any suitable cell in which chemical energy is converted to electric energy by a spontaneous electron transfer reaction. The term includes cells with non-spontaneous reactions, cells with spontaneous reactions, galvanic cells, electrolytic cells and a combination thereof. Optionally, power source can be rechargeable. Optionally, power source can include an external power source. In one embodiment, an external power source can be used in addition to an internal power source, to facilitate an initially high voltage and then can be optionally disconnected from the device.

However, power source need not be limited to one cell, but may include a plurality of the same or different connected electrochemical cells, a plurality of batteries, and/or electronics configured to increase, control, and change phase of the supplied electric current and wherein the power source can be thin and flexible. Electrochemical cell in device in some embodiments provides electrical potential (voltage) to the desired body area of the subject. In some embodiments, the electrical potential may be adjusted to satisfy at least one of the following criteria.

First, the device voltage may be adjusted to enable an iontophoretic delivery or other mechanism of delivery of encapsulated active substance into the body area. Second, the device voltage may be adjusted to minimize the penetration of the active substance through the body, and to maximize the amount into the desired body area. Third, the device voltage may be adjusted to minimize body area irritation, which may result from excessive electric current, passing into and through the body. Further, the device voltage may be initially high to facilitate poration of the bio-membrane, and then adjusted to a lower voltage for electro-transport of the encapsulated substance.

The power source may optionally be located in any suitable position on the device.

Current provided by the electricity generating device, such as a patch may be any suitable form of current, including DC, AC, pulse or other phase form. In some embodiments electricity generating device provides DC current. In some embodiments, the current provided is from about 0.5 μA/cm² to about 500 μA/cm². In some embodiments, power source is attached to substrate base layer with any suitable means, such as, but not limited to adhesive. In some embodiments adhesive may be an acrylic adhesive.

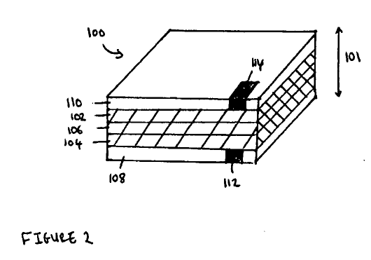

FIG. 2 illustrates a schematic representation of an exemplary power source 100 in accordance with an embodiment of the invention. In some embodiments, power source 100 is thin and flexible. In the embodiment of FIG. 2, the power source is depicted as an electrochemical cell. The thickness 101 of the electrochemical cell 100 may be up to about 4 mm, in some embodiments up to about 2 mm and in some embodiments up to about 1 mm.

In one embodiment, electrochemical cell 100 includes a positive pole layer 102, a negative pole layer 104, and an electrolyte layer 106 interposed therebetween. In some embodiments, electrochemical cell 100 includes one or more additional conductive layers 108 and 110 to improve the conductivity of pole layers 102 and 104. Suitable conductive layers 108 and 110 are in some embodiments made from any suitable conductive material, such as carbon, graphite, silver, platinum or gold or combinations thereof. In some embodiments conductive layers (current collectors) 108 and 110 are graphite or carbon based layers, which can be printed or applied in any suitable way to cell 100. Examples of graphite and carbon based layers include graphite or carbon webs, sheets, inks and cloth. In some embodiments, electrochemical cell includes negative terminals 112 and positive terminals 114, which are in contact with the corresponding pole layer 104 and 102 or with the corresponding conductive layer 108 and 110 or both. Terminals are made of any suitable material such as, but not limited to, graphite or metal and are in some embodiments applied to cell 100 by a suitable printing technology. Terminals may be located in any desired location of cell 100 and may acquire any suitable shape and size, depending on the specific application. Optionally, terminals may protrude from the surface of cell 100.

By way of example, a suitable electrochemical cell 100 is described in U.S. Pat. Nos. 5,652,043, 5,897,522, and 5,811,204, each of which are incorporated herein by reference in their entireties. Briefly, the electrochemical cell described in the above-identified U.S. Patents is an open liquid state, electrochemical cell, which can be used as a primary or rechargeable power source for various miniaturized and portable electrically powered/assisted devices of compact design. In one embodiment, an electrochemical cell 100 may comprise a first layer of insoluble negative pole 104, a second layer of insoluble positive pole 102, and a third layer of aqueous electrolyte 106 disposed between the first 104 and second 102 layers and may include (a) a deliquescent material (not shown) for keeping the open cell wet at all times; (b) an electroactive soluble material (not shown) for obtaining required ionic conductivity; and, (c) a water-soluble polymer (not shown) for obtaining a required viscosity for adhering the first and second layers to the third layer.

Yet, in another preferred embodiment, an electrochemical cell may comprise a plurality of self-contained, serially connected galvanic power sources, as described for example in U.S. Pat. No. 6,421,561, which is incorporated herein by reference in its entirety.

In some embodiments, the power source is applied using a suitable printing technique.

In an alternative embodiment, device electrodes, without the need for an additional power source, may facilitate powering of the cell. For example device electrodes may be configured as a galvanic couple, wherein there is a potential difference between the two electrode materials facilitating a flow of electrons, which can facilitate assisting dermal delivery of a substance. One example of such an electrode galvanic configuration, which can be used in the present invention, is described in US Patent Application Publication No. 2005-0004508 A1.

The Base Member Substrate

In some embodiments, electricity generating device includes a base member substrate, e.g. base layer substrate 22 in FIG. 1. Base member substrate may optionally be manufactured from any suitable material, which can accommodate the device components. Suitable materials include, but are not limited to woven material, non-woven material, polymers, conducting material, non-conducting material, paper, cardboard, plastic, synthetic materials, natural materials, fabric, metals, wood, glass, Perspex, or a combination thereof. In some embodiments, the material of base member is a non-conductive material. In one embodiment, base member is made from polyester. Optionally, base member can be made up of a plurality of materials, which can be stacked or connected in a co-planar way by any suitable attachment means. In some embodiments, base member is made up of one continuous piece of material.

Encapsulated Formulations

Electricity generating device of the present invention may be designed and configured to be used with at least one, and possibly a plurality of external substances. In some embodiments external substances are encapsulated, e.g., encapsulated substance 14 in FIG. 1. In some embodiments, external substance includes at least one active substance. At least one active substance may include a pharmaceutically, cosmetically, or cosmeceutically active substance, a drug, a natural substance, a synthetic substance, a herbal substance, a decorative active substance, such as an ink for a tattoo or permanent make-up or a combination thereof. Optionally external substance can include an inactive substance, an additive or any other substance known in the art which is used in pharmaceutical or cosmetic formulations. The term ‘substance’ as used herein, includes but is not limited to a compound, a formulation, a composition, an active substance, an inactive substance, a natural substance, an artificial substance, any physical form of substance, a chemical, a drug, a cosmetic and a combination thereof. The present invention provides electrically driven delivery of external substances which are encapsulated. In some embodiments, external substances are micro, sub micro or nano encapsulated.

In some embodiments the microencapsulated systems used include, but are not limited to vesicles, rigid vesicles, elastic vesicles, monolayer vesicles and multilayer vesicles, liposomes, niosomes, proniosomes, transfersomes®, ethosomes, L-595-PEG-8-L vesicles, nanoemulsions, nanosomes and nanoparticles and combinations thereof as potential vehicles for the delivery of cosmetics and/or drugs and the optimized disposition of active ingredients in particular bio-membrane layers. In some embodiments, the encapsulated formulations are configured to be delivered to the target region and at the target region the active formulations are released from the encapsulated carriers. In some embodiments, the encapsulated formulation can include a suitable targeting means, for facilitating targeting to a specific body area and releasing the encapsulated substances at the target site.

In some embodiments, wherein delivery to the stratum corneum is desired, rigid vesicle encapsulation may be preferred. In some embodiments wherein delivery to the dermis is required, elastic vesicles may be preferred. In some embodiments, wherein delivery to the stratum corneum and dermis is required, a mixture of elastic and rigid vesicles may be used.

Any suitable liposomal formulation can be used, such as rigid and elastic liposomes, wherein a liposome is a spherical lipid bilayer that has the ability to encapsulate a formulation, which is freely disposed in the interior. Any suitable method of liposomal encapsulation as detailed in the art can be used to encapsulate the active substance/s. In such a way the liposomes can encapsulate hydropholic and hydrophobic compositions, which can be charged, oppositely charged or uncharged. Depending on the requirements of each drug/cosmetic/active substance, the liposome characteristics can be modified. In one embodiment, the size and composition of the spheres can be modified in order to reduce the rate of liposome degradation and therefore slow down the release of the contents. Liposome affinity for a given tissue can also be incremented by varying vesicle composition, electrical charge or by adding receptors or adhesion factors—thereby contributing to increase drug presence in the target tissues or organ.

In most liposome-encapsulated products the substances destined for delivery are introduced within the vesicles in the course of liposome manufacture, for once the latter have been created it is no longer possible to insert anything into them. In contrast, in the DRV technique the Liposomes are produced with concomitant active drug insertion, and the latter therefore remains external to the vesicles.

Nanoemulsions are lipids enclosing a liquid lipid core or oil-in-water emulsions. Lipid nanoparticles are lipid structures enclosing a solid lipid core. In some embodiments they can be added to existing formulations.

Lipid Nanoparticles have a similar structure to Nanoemulsions. Their size ranges typically from 50 to 1000 nm. The difference is that the lipid core is in the solid state. The matrix consists of solid lipids or mixtures of lipids. To stabilize the solid lipid particle against aggregation, surfactants or polymers can be added, whereby natural lecithin are preferred as is the case with Nanoemulsions. If lipid Nanoparticles are intended to be used as a carrier, the active ingredients can be dissolved or finely dispersed in the lipid matrix. Any suitable method of making the encapsulation vesicle as known in the art can be used in the present invention.

In some embodiments the type of encapsulation is chosen depending on factors, which include, but are not limited to the body area region to be treated, the depth of penetration desired, size of vesicle, size and state of active formulation, stability of active formulation, charge desired, speed and rate of delivery to and/or through the bio-membrane and a combination thereof. In an embodiment, wherein deeper penetration depth is required, a smaller vesicle may be preferred, whereas when more superficial penetration is desired a larger vesicle may be used. In some embodiments, the active or inactive compounds/formulations or combinations thereof are encapsulated using standard known techniques. The encapsulation vesicle can then be charged by using standard charging techniques, such as but not limited to use of surfactants, ionization techniques and combinations thereof. In some embodiments, the encapsulation vehicle is uncharged.

In one non-limiting example, wherein an active compound which is to be delivered causes irritation, a mixture of encapsulated vesicles can be prepared, wherein a vesicle, which facilitates quicker delivery, such as a smaller vesicle may include an anti-irritant formulation and a vesicle, which facilitates slower delivery can include the active composition. In such a way, the mixture of encapsulated formulations can be electrotransported, facilitating an initial anti-irritation effect and a later effect by the active composition. In a similar way, a mixture of encapsulated formulations can be electrotransported to facilitate different treatment rates and different times for initial and final effect.

In a further non-limiting example, the present invention can provide a device or kit for treatment of more than one aspect or symptom of a disease or condition. For example, in a condition, such as a wound, which can feature inflammation and infection in different areas of the wound region, a mixture of vesicles can be prepared for electrotransportation, wherein vesicles which are configured for less deep penetration can facilitate treatment of the more superficial sites of inflammation and vesicles which are configured for deeper penetration can facilitate treatment of the deeper sites of infection.

The charged encapsulated formulation can optionally be disposed in a conductive hydrogel or can be disposed without conductive hydrogel onto device electrodes or onto the desired bio-membrane body area or in a retainer which is attached or attachable to the device of the present invention. In an embodiment, wherein encapsulated formulation is applied directly to bio-membrane, a delay period can optionally be waited before application of device of the present invention. Optionally, a delay period can facilitate a pretreatment with the encapsulated formulation, without the influence of the electricity generating device.

In some embodiments, after treatment with the device of the present invention and encapsulated formulation, a second amount of encapsulated formulation can be applied to the bio-membrane region which has been treated.

In an alternative embodiment, the bio-membrane can be treated with the electricity generating device of the present invention without encapsulated formulation. After the device has been removed, encapsulated formulation can be applied to the bio-membrane which has been electrically stimulated.

In some embodiments, wherein the encapsulated formulation is positively charged, it can be placed on the anode and when the device is contacted with the bio-membrane, the current facilitates delivery of the charged encapsulated formulation. In some embodiments, the charged vesicle is viable/stable with the active compounds contained within until contact with the inner bio-membrane layers or viable skin layers. The actives are then released to the target area.

Conductive Formulations

In some embodiments, the electricity generating device of the present invention includes a conductive fluid/composition, e.g., conductive substance 12 in FIG. 1. In some embodiments, the conductive fluid/composition facilitates a conductive interface between the device and bio-membrane. Device of the present invention may be designed and configured to be used with at least one and possibly a plurality of external substances including at least one encapsulated substance. In some embodiments, device of the present invention may be designed and configured to be used with a mixture of encapsulated and non-encapsulated substances. Such encapsulated substances, described in detail herein, may be designed to be contained in a conductive fluid/substance, also described in detail herein. The term ‘conductive fluid’ as used herein includes the terms ‘conductive substance’ and ‘conductive composition’ and includes any suitable liquid, semi-solid or solid form of conductive material. The encapsulated substance/s can optionally be included in the conductive fluid/composition or can be added to the conductive fluid/composition before use of the device.

Conductive fluid/composition may be an electrically conductive and adhesive hydrogel, suitable for use as a skin contact adhesive and, particularly, suitable for use as an electrical interface for electrodes of the device. The hydrogels are polymeric acrylates and may be, for example, made from acrylic esters of quatemry chlorides and/or sulfates or acrylic amides of quaternary chlorides. They can be formed by free radical polymerization in the presence of water, preferably by ultra-violet curing with initiator and multi-functional cross-linking agent. The hydrogel may include a buffer system to help prevent discoloration of the hydrogels and/or hydrolysis of the hydrogels and/or to improve shelf-life.

Other additives may be incorporated into the present hydrogels either before or after curing (e.g., conductivity enhancers, pharmaceuticals, humectant plasticizers, etc.) depending on intended end-use. An additive that may be added to the hydrogel is a conductive adhesive matter (additive) that serves to allow the conductive fluid/composition to both attach device, such as patch to the skin of the subject and to serve as the conductive interface between the electrode and the skin. The adhesive additive may be a polymeric adhesive and may be pressure or temperature activatable or it may be activated by the exposure to the ambient atmosphere.

In some embodiments, the hydrogel is sufficiently cohesive, yet remains readily separable. Optionally, the hydrogel may be in sheet form. Further details pertaining to hydrogels suitable for use in the context of the present invention are described in, for example, U.S. Pat. No. 5,800,685, which is incorporated herein by reference in its entirety.

In any case, an aqueous conductive fluid/composition in accordance with the teachings of the present invention may include water, alcoholic/aqueous solutions, at least one salt or any other charged agent and may further include a buffering medium.

It is appreciated that non-aqueous conductive fluid/compositions may also be employed.

The conductive fluid/compositions used in conjunction with the electricity generating device of the present invention are in some embodiments administered by deposition on one or both device electrodes either using a retainer or without a retainer. It is appreciated that the conductive fluid/composition may alternatively or in addition be administered by topical application to the bio-membrane, such as, but not limited to skin. The term “topical” is used herein to refer to administration of a substance on the surface of the bio-membrane, such as skin or mucosal tissue, which can be applied via direct application (e.g., spreading), via an impregnated porous material or object or by spraying or misting. In some embodiments, wherein the conductive substance is in sheet form, the substance can be placed directly on the skin.

Retainers

Retainers, e.g., holding means/retainer 22 in FIG. 1, used with the device or kit of the present invention may vary in shape, size and method of dispensing according to the quantity, application and location relevant to the treatment. In some embodiments retainer is a separator. The use of the term “separator” is intended to describe a retainer made of a porous non-conductive material, such as a sponge, paper, etc., that serves to retain the conductive fluid/composition therein. Separators offer advantages over other retainers in that they allow precise positioning of the conductive fluid/composition, they are not messy, and they permit a precise dosage to be administered. The term ‘separator’ also includes a substantially solid, semi-solid or sheet conductive composition/material, such as a hydrogel in sheet form. In some embodiments, wherein the separator is a conductive composition, the separator may be configured into a desired shape, such as for example by cutting, wherein the shape may be configured to facilitate no electrical contact between the electrodes. The shape may also be configured to facilitate area and type of treatment.

Conductive fluid/composition may be retained in a retainer/separator in such a manner that objects that are in contact with the separator are also in contact with the fluid/composition contained therein. Accordingly, electrical contact may be made with the conductive fluid/composition held within a separator by establishing physical contact between the electrode and the separator.

Separators may be designed and configured to fit between one or both of device electrodes and the bio-membrane, such as skin of the subject, thus providing a simple, clean and convenient electrode/skin interface through which electricity may flow via the conductive fluid/composition to the area of treatment. As stated earlier, in some embodiments separators are constructed so that their non-conductive structure does not impede the electrical contact between device electrodes and the conductive fluid/composition therein. In some embodiments, a separator will be positioned such that it or its contents do not create an electrical contact between device electrodes. Separators may be fabricated in the form of plugs, cartridges or tablets and the like which are designed to be compatible with different shapes, sizes and configurations of device electrodes. In some embodiments, separator, may be a thin waferlike container, which may be of a desired shape to be compatible with both the area of treatment and the electrode in use. Such separators may be protected by a thin film layer, which will be peeled off immediately prior to use.

Separators may be packaged for storage or use as may be compatible with any particular embodiment of the kit of the present invention. Separators may be individually packaged in order to preserve shelf life and to avoid evaporation of the conducting fluid/composition and/or substance contained therein. In some embodiments, separator includes conductive fluid/composition and encapsulated active substance. In some embodiments, encapsulated active substance is not included in separator and is applied prior to use of patch. Encapsulated active substance can optionally be applied to separator, applied topically to skin or applied directly onto device electrodes.

The use of the above described retainers, particularly separator, is intended to render an electricity generating device of the present invention extremely user friendly and almost foolproof in its employment. Some embodiments of the invention may have separator as the vehicle for the conductive fluid/composition, which can be positioned with precision on either the electrode or on the bio-membrane, such as skin of the subject. A retainer which is a sheet hydrogel or other conductive composition, cut into the shape of the electrode can be facilely applied with precision onto the device.

It is appreciated that the precise positioning of the conductive fluid/composition, either upon the relevant electrode or upon the bio-membrane, such as skin of the subject, is critical to the effective conduction of electric current through the skin of the subject. Accordingly, a kit comprising device and one or more of retainers including encapsulated active substance/s may also contain any other implements, instruction, markings, aids or devices that will serve to assist a user to properly apply and position the conductive fluid/composition as required.

The conductive fluid/composition may be designed to be retained in at least one, or possibly many, retainers.

Kit

In some embodiments a combination of device and at least one retainer may form a kit that may be retained by a patient for use for a variety of applications. In an embodiment wherein the encapsulated active substance is not contained in the conductive fluid/composition, the kit can feature a combination of device, retainer/separator and encapsulated active substance. In an embodiment wherein the encapsulated active substance is included in the conductive fluid/composition, the kit features a combination of device and conductive fluid, wherein the conductive fluid includes encapsulated active substance. Optionally, encapsulated active substance can be applied directly to skin or can be added to conductive/fluid composition, prior to application of the conductive fluid/composition. The kit may readily facilitate a disposable or, reusable device, which can be used with a variety of encapsulated active ingredients, different doses and different shapes and sizes of conductive fluid/substance and/or retainers depending on the use of the device and kit.

Active Ingredients

In some embodiments of the present invention, the electricity generating device may transdermally or intradermally deliver a pharmaceutical substance, a cosmetic substance or a cosmeceutical substance. As used herein, the terms “transdermal” and “intradermal” and grammatical variations thereof, respectively refer to the delivery of a composition through/across a bio-membrane such as the skin. As used herein, the term “pharmaceutical” refers to preventative, therapeutic and anesthetic substances. Therapeutic, as used herein, is understood to include any substance serving to prevent, cure, heal, treat medically or preserve health. Anesthetic, as used herein, is understood to include any substance serving to cause a loss of tactile sensation, particularly pain. Such substances, may be in the form of uncharged or charged molecules which will respond to an electric current. It is appreciated that any pharmaceutical substance, cosmetic substance or cosmeceutical substance may be delivered by the invention described herein.

According to the present invention the substance is encapsulated in any suitable encapsulation carrier vehicle. Description of suitable encapsulation carrier vehicles is described herein.

In general, ‘substance’ includes therapeutic substances, or drugs, or active substances/ingredients/compositions in all of the major therapeutic areas including, but not limited to, antiinfectives such as antibiotics and antiviral agents, and antifungal agents, analgesics including fentanyl, sufentanil, buprenorphine and analgesic combinations, anesthetics, anorexics, antiarthritics, antiasthmatic agents such as terbutaline, anticonvulsants, antidepressants, antidiabetic agents, antidiarrheals, antihistamines, antiinflammatory agents, antimigraine preparations, antimotion sickness, preparations such as scopolamine and ondansetron, antinauseants, antineoplastics, antiparkinsonism drugs, cardiostimulants such as dobutamine, antipruritics, antipsychotics, antipyretics, antispasmodics, including gastrointestinal and urinary, anticholinergics, sympathornimetics, xanthine derivatives, cardiovascular preparations including calcium channel blockers such as nifedipine, beta-blockers, beta-agonists such as salbutamol and ritodrine, antiarrythmics, antihypertensives such as atenolol, ACE inhibitors, diuretics, vasodilators, including general, coronary, peripheral and cerebral, central nervous system stimulants, cough and cold preparations, decongestants, diagnostics, hormones such as parathyroid hormone, growth hormone and insulin, hypnotics, immunosuppressives, muscle relaxants, parasympatholytics, parasympathomimetics, anti-oxidants, nicotine, prostaglandins, psychostimulants, sedatives and tranquilizers.

The invention is also useful for the delivery of cosmetic and cosmeceutical substances. Such substances, include, for example, skin acting anti-oxidants, such as caretenoids, ascorbic acid (vitamin C) and vitamin E, as well as other vitamin preparations and other anti-oxidants, anti wrinkling agents such as retinoids, including retinol (vitamin A alcohol), alpha-hydroxic acids, beta-hydroxy acid, better known as salicylic acid, combination-hydroxy acids and poly-hydroxy acids, and hydrolyzed and soluble collagen and others, moisturizers such as hyaluronic acid and others, anticellulite agents such as aminophyllines and others, skin bleaching agents such as retinoic acid, hydroquinone and peroxides and others, botanical preparations such as extracts of aloe-vera, wild yam, hamamelitanin, ginseng, green tea and others.

Means for Maintaining Viability/Stability of Encapsulated Composition Prior to Bio-Membrane Penetration

A use of the device and/or kit of the present invention is to deliver at least one active substance in an encapsulated formulation to and/or into and/or through a bio-membrane. It is advantageous that the encapsulated composition maintains its encapsulated state until it has been delivered to the target body region and/or penetration depth, where the actives can be released. In some embodiments, device and/or kit includes a means for maintaining stability of encapsulated composition prior to bio-membrane penetration. The term ‘stability of encapsulated composition/s’ as used herein refers to the viability, stability and maintaining of the intact encapsulation vehicle. The term includes the encapsulation vesicle maintaining its structure without releasing its contents and/or without any substantial degradation or disintegration or break up. Means for maintaining viability or stabilizing the encapsulated composition/s, include a direct contact separator to facilitate a separation between the electrode and encapsulated composition/s, current control, low current, low charge on encapsulating carrier vehicle, heat control, cooling element and a combination thereof.

A direct contact separator can be any suitable conductive means, which can facilitate preventing direct contact between the electrodes and encapsulated formulation.

Current control means can facilitate control on the current at the active electrode, which can be changed according to the charge on the encapsulation carrier. In some embodiments low voltage can be used.

Heat control means and cooling elements can facilitate suitable temperature for stability/viability of encapsulation carriers, such as liposomes.

Uses

The electricity generating device of the present invention can be effective in the treatment and prevention of any suitable condition including a skin condition or other medical condition or cosmetic condition such as, but not limited to, acne treatment, sebum regulation, rosacea, age spots, dermatitis, skin and nail viral, fungal and bacterial infections, onychomycosis, Cellulitis, Acute Lymphangitis, Lymphadenitis, Erysipelas, Cutaneous Abscesses, Necrotizing Subcutaneous Infections, Staphylococcal Scalded Skin Syndrome, Folliculitis, Furuncles, Hidradenitis Suppurativa, Carbuncles, Paronychial Infections, Erythrasma, disorders of the hair follicles and sebaceous glands, Perioral Dermatitis, Hypertrichosis, Alopecia, Pseudofolliculitis Barbae, Keratinous Cyst scaling disease, dark rings under the eyes, scars, wounds, cellulite treatment, skin and tooth whitening, pigmentation disorders, sun damaged skin, fine facial lines, laugh lines, aging skin, dry skin, wrinkles, puffy eyes, lifting skin, folliculitis, dermatitis, Contact Dermatitis, Atopic Dermatitis, Seborrheic Dermatitis, Nummular Dermatitis, Chronic Dermatitis Of The Hands And Feet, Generalized Exfoliative Dermatitis, Stasis Dermatitis, psoriasis, warts, herpes, benign tumors, malignant tumors, pain management, bone healing, facilitating muscle contraction, promoting metabolic processes, increasing blood flow, hair growth disorders, treating hyperhidrosis, body decoration, vaginal candidiasis and vaginosis, genital herpes, anaesthesia and a combination thereof.

Methods of Use

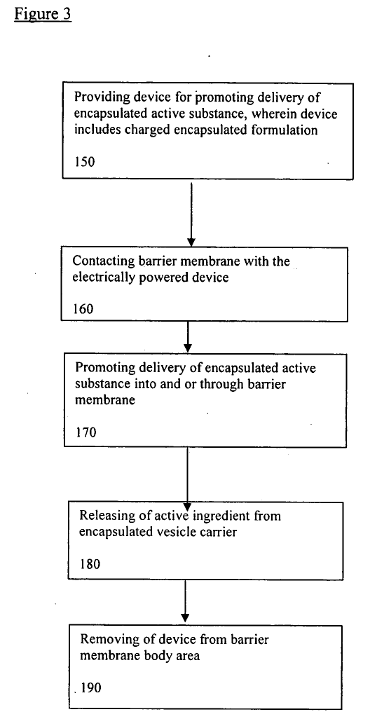

FIG. 3 is a flow chart of an exemplary method of use of a device according to embodiments of the present invention. The flowchart applies to a method of use of a fully integrated patch device for promoting delivery of an encapsulated active substance/s. An electrically powered/assisted device, such as a patch as herein described may be provided 150. In some embodiments, the device includes at least one first electrode, and at least one second electrode and at least one power source, supported on a base member substrate in spaced relation to each other to define a gap therebetween and a holding means for accommodating a conductive encapsulated active formulation and a conductive adhesive layer. The patch may be configured to facilitate providing an electrical current and delivering an encapsulated active agent.

In an embodiment, wherein the patch includes a protective liner, protective liner may be removed from the patch. The subject may contact a bio-membrane of a body area to be treated with the device, which includes the encapsulated active formulation 160. In some embodiments, device is a thin and flexible device, which conforms to the contours of the body and which includes attachment means, for ready attachment to the body area to be treated.

In some embodiments, the contact of the device with the body area facilitates current flow and promotes delivery of encapsulated active agent 170. In some embodiments, the active formulation is released from the encapsulated vesicles in the viable bio-membrane layers 180. Body area region can optionally be treated by electrical stimulation and by active agent.

The device can be removed from the body area at the end of treatment time 190. Time of treatment can vary. The device is in some embodiments removed from contact with the body area after a time period, which can optionally be predetermined or is determined according to the desired dosage, the time it takes for the electrode to be depleted, or until sufficient effect or no more improvement can be seen.

In some embodiments a pretreatment can be applied prior to use of the device. Non-limiting examples of pretreatments include applying a cleanser, applying a moisturizing composition, applying a formulation comprising a pharmaceutically active ingredient, wherein the formulation can be encapsulated or non-encapsulated and the same or different from the active formulation applied with the device, applying a formulation comprising a cosmetic ingredient, applying an antiseptic, desensitizing the body area, such as with an anaesthetic, restyling bio-membrane (cutting, filing, shaping etc), applying a permeation enhancer, microporation of bio-membrane, massaging, or a combination thereof.

In some embodiments a post treatment can be applied to the body area after application of the device. Non-limiting examples of post treatments include applying an occlusion formulation, applying a cleanser, applying a moisturizing composition, applying an anti-irritant, applying a formulation comprising a pharmaceutically active ingredient, applying a formulation comprising a cosmetically active ingredient, wherein the formulation with the active ingredient can be encapsulated or non-encapsulated, and the same or different, from the encapsulated active formulation delivered by the device or a combination thereof.

The treatment can optionally be a one-time treatment or can be repeated in suitable time intervals any suitable number of times. Use of the present invention can facilitate temporary or permanent alleviation and elimination of the above conditions. Duration of effect can be affected by time and frequency of application, type of encapsulation vesicle used, dose of active agent, type and amount of current used and severity of condition. In one embodiment, the patch device is configured for home use. In other embodiments, the patch device can be applied in a supervised environment.

Electro-Transportation of Encapsulated Mixture

The use of electro-transportation for delivery of a mixture of compounds, wherein the mixture includes a certain ratio of compounds, can facilitate delivery of the mixture of compounds into a bio-membrane. However, due to the difference in compound size and charge and state, the penetration, penetration depth and ratio of the different compounds in the mixture, which is actually delivered in the bio-membrane, is not necessarily the same for all the compounds in the mixture. The use of electro-transportation of encapsulated formulations provides a solution to this latent deficiency of electro-transportation. The encapsulated vesicles can be prepared with a pre-determined fixed ratio of any mixture of compounds and electro-transport of the intact vesicles facilitates a method of achieving the same penetration, penetration depth and maintains substantially the same fixed ratio of delivered compounds as in the original prepared encapsulated mixture, for all the compounds/compositions/drugs/cosmetics in the mixture. ‘Mixture of compounds’ can include, but is not limited to same compounds, different compounds, different size compounds, same size compounds, different charged compounds, same charged compounds, same amount of charged compounds, different amount of charged compounds, different physical states of compounds, same states of compounds, compounds with different hydrophobicity, immiscible compounds, miscible compounds, compounds with different or the same therapeutic and/or cosmetic properties and a combination thereof.

The penetration depth and rate of penetration can be controlled by factors, which include, but are not limited to choice of vesicle, such as rigid or elastic vesicles, size of vesicles, charge on vesicles, device current and voltage and a combination thereof. Electro-transport of encapsulation vesicles can facilitate dermal delivery, transdermal delivery, intradermal delivery, delivery to the stratum corneum, delivery to the epidermis, topical delivery to and/or through a bio-membrane and a combination thereof.

The original determined ratio of compounds in a mixture may be delivered in the same ratio to the target body area, as is provided by the present invention. The ratio of compounds may be calculated and fixed to achieve a particular effect. In some embodiments, the ratio of compounds may be important to prevent an adverse reaction or to prevent overdosing by one of the compounds or under-dosing by a compound. In some embodiments, the ratio of compounds may facilitate more than one effect. In some embodiments, the ratio of compounds may facilitate a synergistic effect.

It can be advantageous that all components of a mixture are delivered to the same penetration depth as is provided by some embodiments of the present invention. In such a way, the effect of all components of the mixture is on the same target body area region. Further, if the same ratio of components in a mixture is delivered, but the components do not penetrate to the same depth, the ratio of components delivered may not necessarily facilitate the combined effect of the predetermined ratio of the mixture at the target body area, but each component may separately effect the different body area region to which it has been delivered.

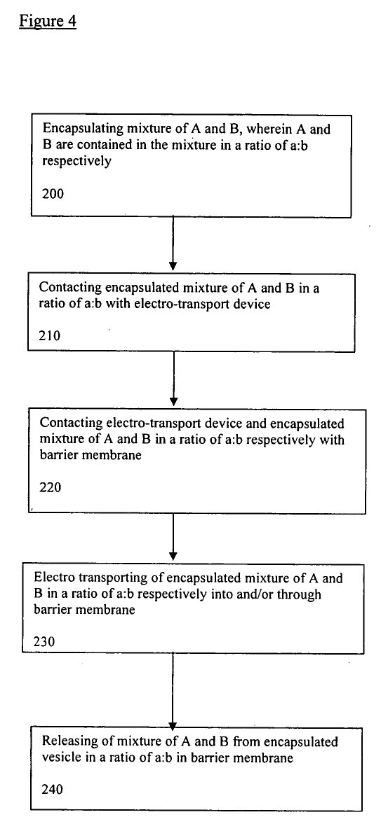

FIG. 4 is a flow chart of an exemplary method of electro-transport delivery of a fixed predetermined ratio of compounds from a mixture of compounds. Any suitable number of compounds/substances/drugs can be in the mixture. The flowchart applies to a method of use of a non-fully integrated patch device or kit for promoting delivery of a fixed ratio of compounds from a mixture of compounds. Substances A and B are encapsulated as a mixture in a ratio of a:b respectively 200. Encapsulated mixture of A and B in a ratio of a:b can be contacted in any suitable way with electro-transport/electricity generating device 210. In some embodiments, encapsulated mixture is applied onto at least one electrode. In some embodiments, encapsulated mixture is disposed in a retainer on active electrode. In some embodiments, encapsulated mixture is applied to bio-membrane. In some embodiments, encapsulated mixture is integrally formed with device. In some embodiments, encapsulated mixture of A and B in a ratio of a:b, is part of a kit including device and encapsulated mixture.

In some embodiments, device and encapsulated mixture of A and B in a ratio of a:b are contacted with bio-membrane 220. Encapsulated mixture of A and B in a ratio of a:b can be electrically delivered into bio-membrane 230.

In some embodiments, the mixture of compounds A and B are released from the encapsulated vesicles in the viable bio-membrane layers in a ratio of a:b 240. In some embodiments, the penetration depth of both compounds A and B is the same.

A similar method can be applied to a fully integrated device, without the need for 210 contacting the encapsulated mixture with the electricity generating device.

Reference is now made to the following experiment, which together with the above description illustrates the invention in a non-limiting fashion.

Experiment

The experiment was conducted to compare delivery of MAP using encapsulated and non-encapsulated MAP

Materials

1. Magnesium L-Ascorbyl-2-Phosphate (MAP) is a negatively charged molecule.

Chemical structure:

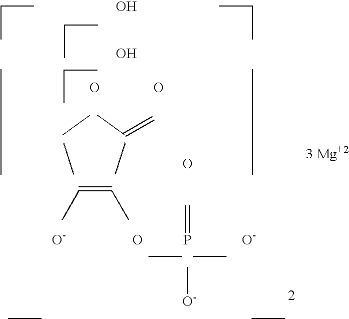

Molecular weight: Mw=228 g/mol

Freely soluble in water p0 2. Whitesphere is a water dispersion of non-ionic nanovesicles (100 nm) that contain MAP.

Experimental Procedure

A tape stripping method was used to evaluate penetration depth.

Passive and iontophoretic experiments were performed in vertical diffusion cells (LGA, Berkeley), in which the skin membrane with an exposed area of 0.64 cm² separates the physically- and electrically-isolated anode and cathode chambers from the receiving compartment. Power supplies, specially designed and manufactured by Power Paper, attached to two Avometers and Silver-Silver Chloride electrodes, produced by Power Paper, were used in the studies. Current and voltage were recorded manually.

In one experiment to determine delivery of non-encapsulated MAP, 3% MAP in ddw was placed in a diffusion cell's cathode donor chamber. Physiologically buffered saline (PBS) at pH 7.4 was placed in the anode donor chamber as well as in the receiving compartment.

To determine delivery of encapsulated MAP, 30% Whitespheres in ddw was placed in the diffusion cell's anode donor chamber and PBS was placed in the cathode donor chamber.

At the end of the treatment period, the entire content of the receiving compartment was drained and the solution was reserved for subsequent analysis of MAP. For the tape stripping procedure, the active formulation and the PBS was removed from the donor compartments; the skin was then separated from the diffusion cell, and the surface was carefully cleaned and dried using cotton balls dampened with PBS. Subsequently, the stratum corneum (SC) beneath the application area was separated by repeated adhesive tape-stripping (20 strips were removed). The tape-strips were separated into three groups: tape-strip 1 in the first group, tape-strips 2-10 in the second group and tape-strips 11-20 in the third group. The compound was extracted from the tape-strips and assayed to yield a total uptake of the active agent into the SC (stratum corneum).

Results

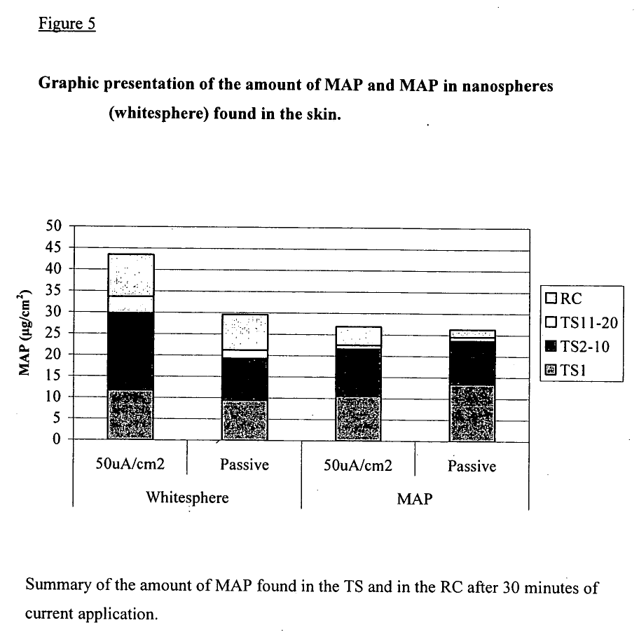

It can be seen from the graph in FIG. 5, that there is significantly greater active delivery of the encapsulated MAP (whitesphere) into the stratum corneum than the non-encapsulated MAP. As such, it can be concluded that encapsulation facilitates increased delivery.

It will be appreciated by persons skilled in the art that the present invention is not limited to what has been particularly shown and described hereinabove. Rather, the scope of the present invention is defined by the appended claims and includes both combinations and subcombinations of the various features described hereinabove as well as variations and modifications thereof which would occur to persons skilled in the art upon reading the foregoing description. Accordingly, it is intended to embrace all such alternatives, modifications and variations that fall within the spirit and broad scope of the appended claims.

What is claimed is:

1. An integrated electricity generating device for delivery of an encapsulated composition to a bio-membrane, comprising:
(a) an electricity generating device;
(b) at least one composition in an encapsulated carrier vehicle in contact with the electricity generating device; and
(c) means for maintaining stability of an encapsulated composition before bio-membrane penetration.

2. The integrated electricity generating device of claim 1, wherein the electricity generating device is selected from the group consisting of an electro-transportation device, iontophoresis device, a bio-membrane patch, a dermal patch, a galvanic stimulation device, an electrokinetic device, an electroporation device, a microneedle, a TENS device, an ultrasound device and a combination thereof.

3. The integrated electricity generating device of claim 2, wherein the dermal patch comprises
at least one active electrode,
at least one counter electrode; and
a power source for supplying power to the device.

4. The integrated electricity generating device of claim 3, wherein the power source is a flexible thin layer electrochemical cell.

5. The integrated electricity generating device of claim 4, wherein the flexible thin layer electrochemical cell is an open liquid state electrochemical cell which comprises a first layer of insoluble negative pole, a second layer of insoluble positive pole and a third layer of aqueous electrolyte, the third layer being disposed between the first and second layers and including:
(a) a deliquescent material for keeping the open cell wet at all times;
(b) an electroactive soluble material for obtaining required ionic conductivity; and
(c) a water-soluble polymer for obtaining a required viscosity for adhering the first and the second layers to the third layer.

6. The integrated electricity generating device of claim 1, wherein the electricity generating device facilitates one of the group consisting of electrical stimulation, electrical driven delivery of a substance, introduction of current and voltage to a bio-membrane and a combination thereof.

7. The integrated electricity generating device of claim 1, wherein the at least one composition is selected from the group consisting of a pharmaceutical, a cosmetic, a cosmeceutical, a formulation additive, an active formulation, an inactive composition, a charged composition, an uncharged composition, a decorative formulation and a combination thereof.

8. The integrated electricity generating device of claim 7, wherein the at least one composition is a plurality of compositions.

9. The integrated electricity generating device of claim 8, wherein the plurality of compositions is a mixture of same charged compositions, oppositely charged compositions, uncharged compositions, a mixture of charged and uncharged compositions, hydrophilic compositions, hydrophobic compositions, mixtures of hydrophilic and hydrophobic compositions and a combination thereof.

10. The integrated electricity generating device of claim 1, wherein the encapsulating vehicle carrier is selected from the group consisting of a micro-encapsulated vehicle, a sub micro encapsulated vehicle, a nano-encapsulated vehicle and a combination thereof.

11. The integrated electricity generating device of claim 1, wherein the encapsulating carrier vehicle is selected from the group consisting of vesicles, rigid vesicles, elastic vesicles, monolayer vesicles, multi-layer vesicles, liposomes, niosomes, proniosomes, transfersomes®, ethosomes, L-595-PEG-8-L vesicles, nanoemulsions, nanosomes, nanoparticles and a combination thereof.

12. The integrated electricity generating device of claim 1, wherein the encapsulating carrier vehicle is selected from the group consisting of a positively charged vehicle, a negatively charged vehicle, an uncharged vehicle and a combination thereof.

13. The integrated electricity generating device of claim 1, wherein the encapsulated composition is contacted with the electricity generating device by being disposed under an electrode selected from the group consisting of positive electrode, negative electrode and combination thereof.

14. The integrated electricity generating device of claim 13, wherein the encapsulated composition is at least one of integrally formed with at least one of the electrodes of the electricity generated device, applied onto at least one of the electrodes, or disposed in a conductive fluid.

15. The integrated electricity generating device of claim 13, wherein the encapsulated composition is separated from direct contact with the electrode by a conductive separator.

16. The integrated electricity generating device of claim 1, wherein the means for maintaining stability of encapsulated composition prior to bio-membrane penetration is selected from the group consisting of a direct contact separator, current control, low current, low charge on encapsulating carrier vehicle, heat control, cooling means and a combination thereof.

17. The integrated electricity generating device of claim 1, wherein the bio-membrane is selected from the group consisting of skin, mucosa, eye membrane, nail, hair, oral membrane, vaginal membrane, rectal membrane and a combination thereof.

18. The integrated electricity generating device of claim 1, for use in the treatment and prevention of a condition selected from the group consisting of a skin condition, acne treatment, sebum regulation, rosacea, age spots, dermatitis, skin and nail viral, fungal and bacterial infections, onychomycosis, disorders of the hair follicles and sebaceous glands, scaling disease, dark rings under the eyes, scars, wounds, cellulite treatment, skin and tooth whitening, pigmentation disorders, sun damaged skin, fine facial lines, laugh lines, aging skin, dry skin, wrinkles, puffy eyes, lifting skin, folliculitis, dermatitis, psoriasis, warts, benign tumors, malignant tumors, pain management, bone healing, facilitating muscle contraction, promoting metabolic processes, increasing blood flow, hair growth disorders, treating hyperhidrosis, body decoration, vaginal candidiasis and vaginosis, genital herpes, anaesthesia and a combination thereof.

19. The integrated electricity generating device of claim 1 as part of a kit.

20. A method of delivering a fixed ratio of compounds from a mixture of compounds to and/or into and/or through a bio-membrane comprising the step of electro-transporting an encapsulated mixture of compounds, wherein the mixture of compounds includes a predetermined fixed ratio of compounds.

21. The method of claim 20, wherein the mixture of compounds is selected from the group consisting of same compounds, different compounds, different size compounds, same size compounds, different charged compounds, same charged compounds, same amount of charged compounds, different amount of charged compounds, different physical states of compounds, same states of compounds, compounds with different hydrophobicity, immiscible compounds, miscible compounds, compounds with different or the same therapeutic and/or cosmetic properties and a combination thereof.

22. The method of claim 20, wherein the electro-transporting includes delivery of a ratio of compounds that is the same as the fixed ratio of compounds encapsulated.

23. The method of claim 20, wherein the electro-transporting is facilitated by an electricity generating device selected from the group consisting of an electro-transportation device, iontophoresis device, a bio-membrane patch, a dermal patch, a galvanic stimulation device, an electrokinetic device, an electroporation device, a TENS device, an ultrasound device and a combination thereof.

24. The method of claim 20, wherein the electro-transporting includes delivery of each of the compounds in the encapsulated mixture to the same penetration depth.

25. The method of claim 24, wherein the penetration depth is controlled by factors selected from the group consisting of type of encapsulation vesicles, rigid or elastic vesicles, size of vesicles, charge on vesicles, device current and voltage and a combination thereof.

26. An integrated electricity generating device for delivery of a fixed ratio of compounds in a mixture to a target site, comprising:
(a) an electricity generating device; and
(b) a mixture of compounds in a fixed ratio in an encapsulated carrier vehicle in contact with the electricity generating device;
wherein the electricity generating device promotes delivery of the encapsulated mixture to the target site and facilitates delivery at the target site of the fixed ratio of compounds in the mixture.

27. An electricity generating device for delivery of a mixture of compounds to the same penetration depth, comprising:
(a) an electricity generating device; and
(b) a mixture of compounds in an encapsulated carrier vehicle in contact with the electricity generating device;
wherein the electricity generating device promotes delivery of the encapsulated mixture of compounds to a target site and facilitates delivery of the mixture of compounds to the same penetration depth.

28. The electricity generating device of claim 27, wherein the encapsulated carrier is configured to release the mixture of compounds at the target site.
